(19) United States
(12) Patent Application Publication
(10) Pub. No.: US 2011/0189121 A1
(43) Pub. Date: Aug. 4, 2011

(54) BITABLETS COMPRISING COMPACTED POLYALLYLAMINE POLYMER AND METHOD FOR THE PRODUCTION THEREOF

(75) Inventors: Maria Genth, Ulm (DE); Max-Werner Scheiwe, Maulburg (DE)

(73) Assignee: ratiopharm GmbH, Ulm (DE)

(21) Appl. No.: 13/001,242

(22) PCT Filed: Mar. 20, 2009
(86) PCT No.: PCT/EP2009/002071

§ 371 Date: Mar. 8, 2011

(30) Foreign Application Priority Data
Jun. 25, 2008 (DE) 10 2088 030 046.2

(51) Int. Cl. A61K 31/785; A61P 13/12; B29C 43/02; B29C 43/14

(52) U.S. Cl. 424/78.38; 427/2.14; 264/109; 264/120

(57) ABSTRACT

The invention relates to a method for producing tablets comprising a polyallylamine polymer, comprising the steps (i) preparation of a polyallylamine polymer or pharmaceutically compatible salts thereof, optionally in a mixture with one or more pharmaceutical excipients; (ii) compaction to give a slug; (iii) granulation of the slug; and (iv) compression of the resulting granules to give tablets; and also to tablets, sachets and slugs comprising a compacted polyallylamine polymer. In addition, the invention relates to tablets comprising a polyallylamine polymer, in particular Sevelamer, with a bimodal pore size distribution.

The invention relates to a method of producing tablets comprising a polyallylamine polymer, comprising the steps (i) preparation of a polyallylamine polymer or pharmaceutically compatible salts thereof, optionally in a mixture with one or more pharmaceutical excipients; (ii) compaction to give a slug; (iii) granulation of the slug; and (iv) compression of the resulting granules to give tablets; and also to tablets, granules and slugs comprising a compacted polyallylamine polymer. In addition, the invention relates to tablets comprising a polyallylamine polymer, in particular Sevelamer, with a bimodal pore size distribution.

Sevelamer (INN) is a polyallylamine polymer known in the specialist field which has phosphate-binding properties. The use as medicament was initially described in EP 0 716 606 B1. Sevelamer hydrochloride is available commercially under the name “Renagel®” and is used in particular for dialysis patients with an excess of phosphate in the blood (hyperphosphatemia) for binding phosphate from food.

The formulation of Sevelamer to give commercially available tablets is usually carried out by direct compression.

EP 1 153 940 A1 describes Sevelamer with a density of from 1.18 to 1.24 g/cm³. It was found that Sevelamer with this density can be processed by direct compression to give tablets which have an advantageous hardness. However, it was established that the hardness of the tablets is inadequate if Sevelamer with a density of 1.25 g/cm³ is used, see Table 1 in EP 1 153 940.

EP 1 239 837 B1 likewise discloses the direct compression of Sevelamer to give tablets with a high fraction of active ingredient. It was found that it is particularly important to adjust the water content of the Sevelamer polymer exactly. Good tablet properties were achieved particularly with a water content of from 5 to 7% by weight. If the water content is below 5% by weight, the tablets exhibit an undesirably low hardness. For a water content above 8%, the disintegration time is undesirably extended.

In addition, EP 1 304 104 B1 relates to tablets comprising Sevelamer which have been produced by direct compression. It was found that (depending on the water content of the Sevelamer) tablets with advantageous hardness and disintegration time can only be produced if approximately 30% by weight of crystalline cellulose is added to the active ingredient polymer, see, for example, FIGS. 1 and 2 in EP 1 3 04 104 B1. Tablets with 200 mg of active ingredient and 100 mg of filler were produced.

Finally, WO 2006/050315 A2 describes the production of Sevelamer carbonate formulations, where a tableting likewise takes place by means of direct compression. It was found that the disintegration time after storage for 3 weeks is only acceptable if Sevelamer carbonate is mixed with Sevelamer hydrochloride. However, the addition of Sevelamer hydrochloride reduces the hardness of the resulting tablets, see Table 1.

It can consequently be established that the methods of producing tablets comprising polyallylamine polymer described in the prior art have numerous disadvantages, for example as a result of specific requirements on the water content, the density or the salt composition of the active ingredient, or as a result of the need for specific excipients. It was therefore an object of the invention to overcome the disadvantages which arise in the prior art.

Specifically, it was an object of the invention to provide a method of producing tablets comprising polyallylamine polymer, in particular Sevelamer tablets, the aim being to advantageously process a polyallylamine polymer, in particular Sevelamer, with a variable water content, for example with a water content of from 1 to 14%. In particular, it should be possible to provide tablets both with a rapid disintegration time (less than 15 minutes, preferably less than 10 minutes, in particular less than 8 minutes, e.g. 5 to 7.5 minutes) and also with an advantageous hardness (more than 100 newtons (N), preferably more than 120 newtons, in particular more than 150 newtons), where the water content can vary, can be e.g. 1 to 14%.

It was also an object of the invention to provide a method of producing tablets comprising polyallylamine polymer, in particular Sevelamer tablets, the aim also being to advantageously process a polyallylamine polymer, in particular Sevelamer, with a density greater than 1.24 g/cm³, for example with a density of from 1.25 to 1.30 g/cm³. In particular, it should be possible to provide tablets both with a rapid disintegration time (less than 15 minutes, preferably less than 10 minutes) and also with an advantageous hardness (more than 100 newtons, preferably more than 120 newtons), which comprise Sevelamer with this high density.

Furthermore, it was an object of the invention to provide a method of producing tablets comprising Sevelamer which exhibit advantageous coatability. During the coating of the tablets according to the invention, no “flaking” should arise.

Furthermore, it was an object of the invention to provide a method of producing tablets comprising polyallylamine polymer, in particular Sevelamer tablets, where, for example, lactose and mannitol or modifications thereof can be processed advantageously. Likewise, it should be made possible to use further fillers which constitute an alternative to cellulose. In particular, it should be possible to provide tablets both with a rapid disintegration time (less than 15 minutes, preferably less than 10 minutes, in particular less than 8 minutes, e.g. 5 to 7.5 minutes) and also with an advantageous hardness (more than 100 newtons, preferably more than 120 newtons, in particular more than 150 newtons), which comprise a polyallylamine polymer, in particular Sevelamer, as active ingredient and, for example, lactose and/or mannitol as excipient.

Furthermore, it was an object of the invention to provide a method of producing tablets comprising polyallylamine polymer, in particular Sevelamer tablets, where a high active ingredient fraction can be advantageously processed, for example an active ingredient fraction of from 80 to 95%. In particular, it should be possible to provide tablets with a high active ingredient fraction which have both a rapid disintegration time and also an advantageous hardness.

Moreover, it was an object of the invention to provide a method of producing Sevelamer tablets, where the carbonate salt of Sevelamer should be advantageously processed, preferably as the sole active ingredient without mixing with Sevelamer hydrochloride. In particular, it should be possible to provide tablets with Sevelamer carbonate which have both a disintegration time which remains stable over the time (disintegration time after storage for 3 weeks below 30 minutes, preferably below 15 minutes), and also an advantageous hardness.

The aim was likewise to provide a granule formulation of polyallylamine polymer, in particular Sevelamer, which can be used advantageously for producing a suspension for administration. The granules should readily flow, not separate during storage and permit an exact dosage from single-dose and multi-dose containers.

All of the objects specified above should be effected (e.g. with regard to process costs, industrial safety and environmental protection) as far as possible without the use of alcoholic solvents.

The objects were achieved by compaction of a polyallylamine polymer, in particular by compaction of Sevelamer, to give a slug.

Consequently, the invention provides a slug comprising a polyallylamine polymer, in particular Sevelamer, obtainable by a method comprising the steps (i) providing the polyallylamine polymer or pharmaceutically compatible salts thereof, optionally in a mixture with one or more pharmaceutical excipients and (ii) compaction to give a slug.

In step (ii), the polyallylamine polymer or preferably the mixture of polyallylamine polymer and one or more pharmaceutical excipient(s) is compacted.

The invention further provides a method of producing tablets comprising a polyallylamine polymer, in particular Sevelamer, comprising the steps

(i) providing a polyallylamine polymer or pharmaceutically compatible salts thereof, optionally in a mixture with one or more pharmaceutical excipient(s);
(ii) compaction to give a slug;
(iii) granulation of the slug; and
(iv) compression of the resulting granules to give tablets, optionally with the addition of further pharmaceutical excipients.

The tablets produced by the method according to the invention can optionally be covered with a film in a further, optional step (v).

Tablets and film-coated tablets obtainable by the method according to the invention are likewise provided by this invention.

Finally, the invention encompasses granules, in particular for filling into sachets, comprising a polyallylamine polymer, in particular Sevelamer, obtainable by a method comprising the steps

(i) providing the polyallylamine polymer or pharmaceutically compatible salts thereof, optionally with one or more pharmaceutical excipients;
(ii) compaction to give a slug; and
(iii) granulation of the slug.

Optionally, further excipients may be added during or preferably after step (iii). In particular, excipients for improving flowability, adhesive tendency, disintegration properties, taste and/or wettability are used here.

The resulting granules are preferably used for producing a suspension for administration. It is preferably poured into a suitable package. Examples of packages are bottles, cans or preferably sachets. In the case of bottles or cans, these may contain one daily dose. Alternatively, multiday doses, e.g. a week's dose or a month's dose, may also be filled into bottles or cans.

The method according to the invention for producing tablets comprising a polyallylamine polymer, in particular Sevelamer, is explained in more detail below. The statements relating to steps (i) and (ii) are also used here for producing the slug according to the invention. The statements relating to steps (i) to (iii) are also used here for producing the granules according to the invention.

In step (i) of the method according to the invention, a “polyallylamine polymer” is firstly prepared.

Within the context of this invention, the term “polyallylamine polymer” encompasses a polymer obtainable preferably by the polymerization of monomers which include an allylamine unit or derivatives thereof, such as, for example, alkylated polyallylamine polymers. Within the context of this invention, it is preferably a crosslinked polyallylamine polymer. In particular, the polyallylamine polymer of the present invention is Sevelamer (INN) or Colesevelam (INN), and pharmaceutically compatible salts thereof.

The polyallylamine polymer preferably has phosphate-binding properties. The alkylated polyallylamine polymer preferably has bile-acid-binding properties.

Polyallylamine polymers are known in the prior art and described, for example, in EP 0 716 606 B1. Derivatives of polyallylamine polymers are described, for example, in EP 0 764 174 B1.

The—preferably crosslinked—polyallylamine polymer of the present invention usually has a weight-average molecular weight of from 1000 to 5 million, preferably from 2000 to 2 million, more preferably from 5000 to 1 million, in particular from 10 000 to 250 000 g/mol.

The polyallylamine polymer preferably includes the following repeating structural unit:

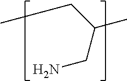

The polyallylamine polymer is preferably crosslinked as a result of the reaction with epichlorohydrin. The crosslinked polyallylamine polymer particularly preferably comprises 5 to 15% by weight, more preferably 9 to 10% by weight, in particular 9.0 to 9.8% by weight, of epichlorohydrin units, based on the total weight of the polymer.

In one preferred embodiment, the crosslinked polyallylamine polymer has the following structure (depicted diagrammatically):

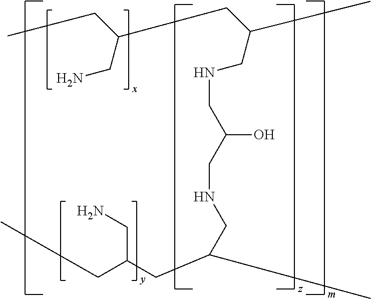

In the above formula, the ratio (x+y):z is preferably 45:1 to 2:1, more preferably 15:1 to 5:1, in particular 9.

In addition, in the above formula, m gives the number of repeating units. Preferably, m is selected such that the number-average molecular weight described above is achieved.

In principle, within the context of this application, the terms “polyallylamine polymer”, “Sevelamer” or “Colesevelam” include both the corresponding polymers and also pharmaceutically compatible salts thereof. These may be one or more salts, which may also be present in a mixture. “Salt” is understood here as meaning that one or more amine groups of the polymer are protonated, in which case a positively charged nitrogen atom is formed which is associated with a corresponding counteranion.

Preferably, the salts used are acid addition salts. Examples of suitable salts are hydrochlorides, carbonates, hydrogencarbonates, acetates, lactates, butyrates, propionates, sulfates, citrates, tartrates, nitrates, sulfonates, oxalates and/or succinates.

In the case of Sevelamer, the pharmaceutically compatible salt is particularly preferably Sevelamer hydrochloride. The pharmaceutically compatible salt is likewise particularly preferably Sevelamer carbonate. Finally, it is particularly preferably a mixture of Sevelamer hydrochloride and Sevelamer carbonate. In one preferred embodiment, this mixture of Sevelamer hydrochloride and Sevelamer carbonate comprises 0.01 to 10% by weight, preferably 0.1 to 5% by weight, of Sevelamer hydrochloride and 90 to 99.99% by weight, preferably 95 to 99.9% by weight, of Sevelamer carbonate, based on the total weight of the mixture.

In one preferred embodiment, 10 to 60%, more preferably 30 to 50%, in particular approximately 40%, of the amino groups are protonated.

In the case of Sevelamer hydrochloride, the following structure (depicted diagrammatically) may be present:

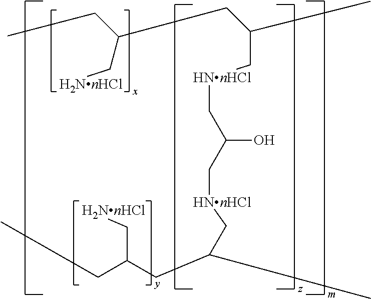

In the above formula, the ratio (x+y):z is preferably 45:1 to 2:1, more preferably 15:1 to 5:1, in particular 9. In addition, in the above formula, m gives the number of repeating units. Preferably, m is selected such that the number-average molecular weight described above is achieved.

Averaged over all units, n is preferably 0.1 to 0.6, more preferably 0.3 to 0.5, in particular approximately 0.4.

In the case of Colesevelam, the pharmaceutically compatible salt is particularly preferably Colesevelam hydrochloride.

In one preferred embodiment, 1 to 90%, more preferably 5 to 50%, in particular 10 to 30%, of the amino groups are alkylated. The alkylation preferably takes place by reacting the polyallylamine polymer with 1-bromodecane and/or (6-bromohexyl)trimethylammonium bromide.

In the case of Colesevelam hydrochloride, the following structure (depicted diagrammatically) may be present:

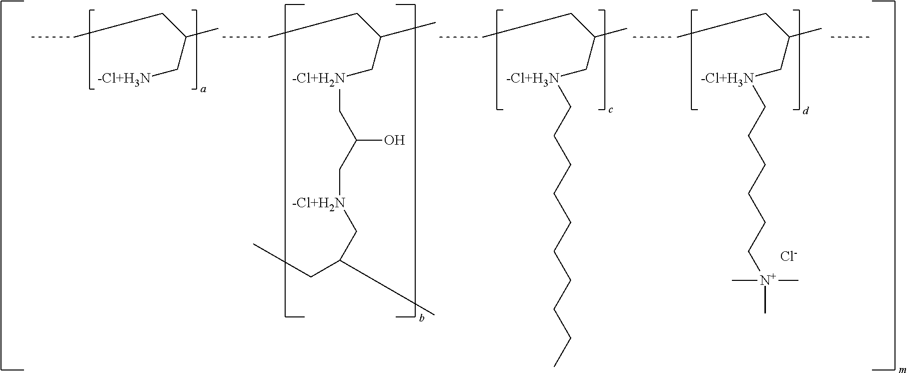

In the formula above, the units (a) are nonalkylated allylamine units, (b) are allylamine units crosslinked with epichlorohydrin, (c) are allylamine units alkylated with a decyl group and (d) are allylamine units alkylated with a (6-trimethylammonium)hexyl group. The fractions of these units add up to 100%, each type being present in the overall polymer preferably in an amount of at least 1%, more preferably at least 5%, in particular at least 10%. Furthermore, the formula does not depict a specific order of the units (a)-(d) since the crosslinking and alkylation of the units occurs in a random manner along the polymer chain. A fraction (preferably less than 10%) of the amines is optionally dialkylated (not depicted). A fraction of the amines (preferably 10 to 90%, in particular 30 to 70%) is optionally protonated. In the formula above, the polymer is shown as hydrochloride. However, instead of chloride, bromide may also optionally be present in the polymer.

In addition, in the above formula, m gives the number of repeating units. Preferably, m is selected such that the number-average molecular weight described above is achieved.

The polyallylamine polymer (or polyallylamine polymer salt) used can comprise water. Usually, it comprises 1 to 15% by weight of water, preferably 2 to 12% by weight of water, based on the total weight of the polymer. In addition, the polyallylamine polymer (or polyallylamine polymer salt) used in the method according to the invention has a density greater than 1.24 g/cm³, preferably a density of 1.25 g/cm³ to 1.30 g/cm³ auf. The expression “density” refers here to the pure density and is determined as described below. In particular, Sevelamer hydrochloride or Sevelamer carbonate with the aforementioned density is used.

In step (i) of the method according to the invention, only the polyallylamine can be prepared. However, in one preferred embodiment, as well as the polyallylamine polymer, one or more pharmaceutical excipient(s) are prepared. These are preferably mixed with the polyallylamine. These are the excipients known to the person skilled in the art, for example those which are described in the European Pharmacopeia.

Examples of excipients are binders, disintegrants, flow regulators, mold release agents, glidants, wetting agents, gel formers, film coatings and/or lubricants.

In one preferred embodiment, polyallylamine polymer is mixed in step (i) of the method according to the invention with one or more fillers and/or binders.

Fillers are generally to be understood as meaning substances which serve to form the tablet body. i.e., fillers produce an adequate tableting mass by “stretching” the active ingredients. Fillers thus usually serve to maintain a suitable tablet size.

Examples of preferred fillers are lactose, lactose derivatives, starch, starch derivatives, treated starch, talc, calcium phosphate, sucrose, sugar alcohols such as mannitol, isomalt, xylitol, sorbitol and/or maltitol; calcium carbonate, magnesium carbonate, magnesium oxide, maltodextrin, calcium sulfate, dextrates, dextrin, dextrose, hydrogenated vegetable oil, kaolin, polymethacrylates, sodium chloride and/or potassium chloride. (Microcrystalline) cellulose or derivatives thereof (e.g. Prosolv®, Rettenmaier & Söhne, Germany) can likewise be used. Furthermore, mixtures of the substances specified above can be used. For example, a spray-dried mixture of lactose monohydrate (preferably 85% by weight) and corn starch (preferably 15% by weight) is preferably used. Such a mixture is commercially available under the trade name “Starlac®”.

Preference is given to using sugar alcohols as fillers, in particular mannitol, isomalt and/or maltitol.

Binders usually serve to increase the strength of the tablets. Binders can generally also contribute to the plastic deformation of the tableting material during compression, e.g. by forming or enlarging the interparticulate surfaces at which bonds can form.

Possible binders are polysaccharides, such as hydroxypropylmethylcellulose (HPMC), carboxymethylcellulose (CMC, in particular sodium salts and calcium salts), ethylcellulose, methylcellulose, hydroxyethylcellulose, ethylhydroxy-ethylcellulose, hydroxypropylcellulose (HPC); guar flour, alginic acid and/or alginates; synthetic polymers such as polyvinylpyrrolidone (Kollidon®), polyvinyl acetate (PVAC), polyvinyl alcohol (PVA), polymers of acrylic acid and salts thereof, polyacrylamide, polymethacrylates, vinylpyrrolidone-vinyl acetate copolymers (copolyvidone), polyalkylene glycols, such as polypropylene glycol or preferably polyethylene glycol, co-block polymers of polyethylene glycol, in particular co-block polymers of polyethylene glycol and polypropylene glycol (Pluronic®, BASF) and mixtures of the specified polymers. If polymeric binders are used, these preferably have a weight-average molecular weight of from 5000 to 120 000 daltons, more preferably from 10 000 to 70 000 daltons.

Examples of preferred binders are gelatin, alginic acid, carbomer, dextrin, ethylcellulose, guar gum, hydroxypropylcellulose, hydroxyethylcellulose, hydroxypropylmethylcellulose, glucose, MgAl silicate, maltodextrin, methylcellulose, polymethacrylate, povidone and derivatives thereof, pregelatinized starch, sodium alginate and/or polyvinyl alcohol (PVA).

It is in the nature of pharmaceutical excipients that these sometimes take on two or more functions in a pharmaceutical formulation. Consequently, individual excipients can for example serve as filler and also as binder.

In one preferred embodiment, in step (i) of the method according to the invention,

(a) 20 to 99% by weight, more preferably 30 to 95% by weight, in particular 40 to 90% by weight, of polyallylamine polymer or pharmaceutically compatible salts thereof and
(b) 1 to 80% by weight, more preferably 5 to 70% by weight, in particular 10 to 60% by weight, of pharmaceutically compatible excipients are mixed.

The mixing can take place in customary mixers. Alternatively, the mixing of active ingredients and excipients can also take place after the granulation step (iii). Alternatively, it is possible that the polyallylamine polymer is mixed with some of the excipients (e.g. 50 to 95%) before the compaction (ii), and that the remainder of the excipients is added after the granulation step (iii). In the case of multiple compaction, the admixing of the excipients should preferably take place before the first compaction step, between two or more compaction steps or after the last granulation step.

The polyallylamine polymer used in step (i) can have a volume-average particle size (d(50)) of, for example, 70 to 400 μm, preferably from 100 to 300 μm.

The polyallylamine polymer used can alternatively be micronized. The micronization preferably takes place before the compaction or before the mixing of the polyallylamine polymer with the excipients. The micronization usually leads to an increase in surface roughness. The micronization takes place, for example, in pin mills or air impact mills. The micronization can also take place by wet-grinding in ball mills. The micronized polyallylamine polymer preferably has a volume-average particle size (d(50)) of from 0.5 to 20 μm, preferably from 1 to 10 μm. The volume-average particle size is determined by means of laser diffractometry (using a Mastersizer 2000 from Malvern Instruments, dispersion module Scirocco 2000 (A) with air as dispersant and 1.5 bar dispersion air pressure; for the calculation, an absorption of 0.1 and a refractive index of 1.52 were used as a basis). The average particle diameter, which is also referred to as D50 value of the integral volume distribution, is defined within the context of this invention as the particle diameter at which 50% by volume of the particles have a diameter which is smaller than the diameter which corresponds to the D50 value. Likewise, 50% by volume of the particles then have a larger diameter than the D50 value. Analogously, the D90 value of the integral volume distribution is defined as the particle diameter at which 90% by volume of the particles have a smaller diameter than the diameter which corresponds to the D90 value.

In step (ii) of the process according to the invention, the polyallylamine polymer from step (i) or preferably the mixture comprising polyallylamine and pharmaceutical excipients from step (i) is compacted to give the slug according to the invention. Here, preference is given to dry compaction.

The compaction preferably takes place in the absence of solvents, in particular in the absence of organic solvents.

The compaction conditions in step (ii) are preferably selected such that the slug has a density of from 1.18 g/cm³ to 1.50 g/cm³, more preferably from 1.19 g/cm³ to 1.40 g/cm³, in particular from 1.20 g/cm³ to 1.30 g/cm³.

The expression “density” refers here preferably to the “pure density” (i.e. not to the bulk density or tamped density). The pure density can be determined using a gas pycnometer. The gas pycnometer is preferably a helium pycnometer, in particular the instrument AccuPyc 1340 helium pycnometer from Micromeritics, Germany, is used.

The compaction is preferably carried out in a roll granulator.

Preferably, the rolling force is 2 to 20 kN/cm, more preferably 3 to 15 kN/cm, in particular 4 to 12 kN/cm.

The gap width of the roll granulator is, for example, 0.8 to 5 mm, preferably 1 to 4 mm, more preferably 1.5 to 3 mm, in particular 1.8 to 2.8 mm.

The compaction device used preferably has a cooling device. In particular, cooling is carried out in such a way that the temperature of the compact does not exceed 55° C.

In step (iii) of the method according to the invention, the slug is granulated. The granulation can take place using methods known in the prior art.

In one preferred embodiment, the granulation conditions are selected such that the resulting particles (granules) have a volume-average particle size (d(50) value) of from 50 to 600 μm, more preferably from 60 to 400 μm, even more preferably 70 to 250 μm, in particular from 80 to 150 μm. The volume-average particle size is determined by means of laser diffractometry (using a Mastersizer 2000 from Malvern Instruments, measurement conditions as described above). In addition, the resulting particles (granules) usually have a d(20) value of the particle size distribution of from 20 to 80 μm, preferably of from 30 to 70 μm, particularly preferably of from 40 to 60 μm. Finally, the resulting particles (granules) usually have a d(90) value of the particle size distribution of from 100 to 800 μm, preferably of from 150 to 600 μm, particularly preferably of from 200 to 500 μm.

In one preferred embodiment, the granulation takes place in a sieving mill. In this case, the mesh width of the sieve insert is usually 0.1 to 5 mm, preferably 0.5 to 3 mm, more preferably 0.75 to 2 mm, in particular 0.8 to 1.8 mm.

The polyallylamine polymers may possibly have an inadequately rough surface, meaning that the compaction step (ii) described above is hindered. Consequently, depending on the nature of the surface, the compaction step (ii) and the granulation step (iii) can be repeated if necessary.

In one preferred embodiment, therefore, the method according to the invention is adapted such that a multiple compaction takes place, the granules resulting from step (iii) being returned one or more times to the compaction (ii).

Preferably, the granules from step (iii) are returned 1 to 5 times, in particular 2 to 3 times.

In the case of multiple compaction, the rolling forces can be up to 25 kN/cm.

In the case of multiple compaction, the granulation (iii) preferably takes place by means of a so-called Frewitt sieve. Sieving is preferably carried out with mesh diameters of from 50 to 250 μm.

In the case of multiple compaction, moreover, it is possible that the amounts of excipients given above are added only partially in step (i), the remaining part amounts being added before the further compaction processes.

In step (iv) of the method according to the invention, the granules obtained in step (iii) are pressed to give tablets, i.e. a compression to give tablets takes place. The compression can take place using tableting machines known in the prior art.

Step (iv) preferably takes place in the absence of solvents, in particular organic solvents, i.e. as dry compression.

In step (iv) of the method according to the invention, excipients can be added to the granules from step (iii).

Examples of suitable excipients are, for example, additives for improving the powder flowability (e.g. disperse silicon dioxide), tablet lubricants (e.g. talc, stearic acid, adipic acid, sodium stearyl fumarate and/or magnesium stearate) and disintegrants (e.g. croscarmellose, crospovidone). Furthermore, the excipients mentioned under step (i) can also be added.

One example of an addition for improving the powder flowability (flow regulator) is disperse silicon dioxide, e.g. known under the trade name Aerosil®. Flow regulators usually have the task of preventing the friction (cohesion) between the individual powder particles or granule grains, and also the adhesion of these to the wall surfaces of the compression device.

Additives for improving the powder flowability are usually used in an amount of from 0.1 to 3% by weight, based on the total weight of the formulation.

Additionally, lubricants may be used. Lubricants generally serve to reduce the sliding friction. In particular, the sliding friction should be reduced which exists during tableting on the one hand between the punches moving up and down in the die bore and the die wall, and also on the other hand between tablet band and die wall. Suitable lubricants are e.g. stearic acid, adipic acid, sodium stearyl fumarate and/or magnesium stearate.

Lubricants are usually used in an amount of from 0.1 to 3% by weight, based on the total weight of the formulation.

Disintegrants is generally the term used to refer to substances which increase the disintegration of an administration form, in particular of a tablet, after it has been introduced into water. Suitable disintegrants are e.g. organic disintegrants such as carrageenan, croscarmellose and crospovidone.

Disintegrants are usually used in an amount of from 0.1 to 10% by weight, preferably from 1 to 5% by weight, based on the total weight of the formulation.

The amount of excipients which is added in step (iv) usually depends on the type of tablet to be produced and on the amount of excipients which has already been added in steps (i) or (ii).

The ratio of active ingredients to excipients is preferably selected such that the resulting tablets comprise

(a) 65 to 99% by weight, more preferably 75 to 95% by weight, in particular 80 to 93% by weight, of polyallylamine polymer or pharmaceutically compatible salts thereof and
(b) 1 to 35% by weight, more preferably 5 to 25% by weight, in particular 7 to 20% by weight, of pharmaceutically compatible excipients.

These quantitative data are particularly preferred when the method according to the invention is used to produce tablets which are swallowed in unchewed form.

The tablets produced by the method according to the invention can therefore be tablets which are swallowed in unchewed form (without a film or preferably covered with a film). These may likewise be chewable tablets or dispersible tablets. Here, “dispersible tablet” is understood as meaning a tablet for producing an aqueous suspension for administration.

In one alternative embodiment, chewable tablets can consequently be produced using the method according to the invention. In this case, the ratio of active ingredients to excipients is preferably selected such that the resulting chewable tablets comprise

(a) 35 to 80% by weight, more preferably 45 to 70% by weight, in particular 55 to 65% by weight, of polyallylamine polymer or pharmaceutically compatible salts thereof and
(b) 20 to 65% by weight, more preferably 30 to 55% by weight, in particular 35 to 45% by weight, of pharmaceutically compatible excipients.

In a further alternative embodiment, dispersible tablets can be produced using the method according to the invention. In this case, the ratio of active ingredients to excipients is preferably selected such that the resulting dispersible tablets comprise

(a) 35 to 60% by weight, more preferably 40 to 55% by weight, in particular 42 to 51% by weight, of polyallylamine polymer or pharmaceutically compatible salts thereof and
(b) 40 to 65% by weight, more preferably 45 to 60% by weight, in particular 49 to 58% by weight, of pharmaceutically compatible excipients.

If granules according to the invention are produced which are preferably used for producing a suspension for administration, the ratio of active ingredients to excipients is preferably selected such that the resulting granules comprise

(a) 25 to 90% by weight, more preferably 40 to 80% by weight, in particular 52 to 74% by weight, of polyallylamine polymer or pharmaceutically compatible salts thereof and
(b) 10 to 75% by weight, more preferably 20 to 60% by weight, in particular 26 to 48% by weight, of pharmaceutically compatible excipients.

The process according to the invention is particularly suitable for producing tablets which comprise a large amount of polyallylamine polymer or pharmaceutically compatible salts thereof.

In one preferred embodiment, the tablets according to the invention comprise 600 mg or more, particularly preferably 800 to 1200 mg, in particular 800 to 1000 mg, of polyallylamine polymer or pharmaceutically compatible salts thereof. This quantitative data is particularly preferred if the method according to the invention is used to produce tablets which are swallowed in unchewed form.

In the case of tablets which are swallowed in unchewed form, it is preferred that these are coated with a film layer. Here, the methods for covering tablets with a film that are customary in the prior art can be used. The ratios of active ingredient to auxiliary that are specified above, however, are based on the uncoated tablet.

For the film coating, preference is given to use macromolecular substances, for example modified celluloses, polymethacrylates, polyvinylpyrrolidone, polyvinyl acetate phthalate, zein and/or shellac.

In this connection, films without an influence on the release of active ingredient, enteric films and slow-release films are possible in principle. Films without an influence on the release of active ingredient are usually water-soluble (preferably, they have a solubility in water of more than 250 mg/ml). Enteric films have a pH-dependent solubility. Slow-release films are not usually water-soluble (they preferably have a solubility in water of less than 10 mg/ml).

Preferred examples of film formers which have no influence on the release of active ingredient are methylcellulose (MC), hydroxypropylmethylcellulose (HPMC), hydroxypropylcellulose (HPC), hydroxyethylcellulose (HEC), polyinylpyrrolidone (PVP) and mixtures thereof. The specified polymers should usually have a weight-average molecular weight of from 10 000 to 150 000 g/mol. Particular preference is given to using HPMC (also referred to as hypromellose), in particular HPMC with a weight-average molecular weight of from 10 000 to 150 000 g/mol and/or an average degree of substitution on —OCH₃ groups of from 1.2 to 2.0.

The layer thickness of the coating is preferably 10 to 100 μm, more preferably 15 to 50 μm or even more preferably 30 to 60 μm.

The tableting conditions in the method according to the invention are also preferably selected such that the resulting tablets have a ratio of tablet height to weight of from 0.005 to 0.3 mm/mg, particularly preferably 0.005 to 0.012 mm/mg.

In addition, the tablets according to the invention preferably have a breaking strength of from 100 to 300 N, particularly preferably from 120 to 200 N, in particular from 140 to 180 N. The breaking strength is determined in accordance with Ph.Eur.6, main edition 2008, 2.9.8.

Moreover, the tablets according to the invention preferably exhibit a friability of less than 2%, particularly preferably of less than 1%, in particular less than 0.5%. The friability is determined in accordance with Ph.Eur. 6.0, section 2.9.7.

Finally, the tablets according to the invention preferably exhibit a disintegration time of less than 15 minutes (min), particularly preferably of less than 10 minutes, in particular less than 8 minutes, e.g. 5 to 7.5 minutes. The disintegration time is determined in accordance with Ph.Eur. 6.0, section 2.9.1 (test A).

The invention provides not only the method according to the invention, but also the tablets produced using this method. It has been found that the tablets produced by this method preferably have a monomodal or bimodal pore size distribution. The invention therefore provides tablets comprising a polyallylamine polymer, in particular Sevelamer or Colesevelam, or pharmaceutically compatible salts thereof, and also optionally pharmaceutically compatible excipients, where the tablet has a monomodal or bimodal pore size distribution. “Bimodal pore size distribution” is understood as meaning that the pore size distribution has two maxima.

The tablet according to the invention arises when the granules from step (iii) are compressed. This compact consists of solid and pores. The pore structure can be characterized in more detail by determining the pore size distribution.

The pore size distribution was determined by means of mercury porosimetry.

Mercury porosimetry measurements were carried out using the porosimeter “Poresizer” from Micromeritics, Norcross, USA. The pore sizes were calculated here on the assumption that the surface tension of mercury is 485 mN/m. From the cumulative pore volume, the pore size distribution was calculated as sum distribution or proportion of the pore fractions in percent. The average pore diameter (4V/A) was determined from the overall specific mercury intrusion volume (Vtotᵢₙₜ) and the total pore area (Atotₚₒᵣ) according to the following equation.

$$4 V / A = \frac{4 \cdot {Vtot}_{int} [ ml / g ]}{{Atot}_{por} [ {m}^{2} / g ]}$$

In one preferred embodiment, the slug according to the invention (obtainable in step (ii) of the method according to the invention) has a pore size distribution maximum of from 5 to 50 μm, more preferably 10 to 30 μm, in particular 11 to 25 μm.

In one preferred embodiment, the granules according to the invention (obtainable in step (iii) of the method according to the invention) have a pore size distribution maximum of from 10 to 100 μm, more preferably 20 to 80 μm, in particular 30 to 60 μm.

In one preferred embodiment, the tablets according to the invention (obtainable in step (iv) of the method according to the invention) have a pore size distribution maximum of from 1 to 10 μm, more preferably 2 to 8 μm, in particular 3 to 6 μm.

In the method according to the invention, therefore, the process parameters described above are preferably selected such that the described pore sizes are achieved.

Finally, the invention provides the tablets according to the invention or granules for treating hyperphosphatemia or hyperlipidemia and also for improving glycemic control.

The invention will be illustrated by reference to the examples below.

### EXAMPLES

### Examples 1.1 to 1.3

### Tablets which are Swallowed in Unchewed Form

The following tablets were produced in accordance with the method of the invention.

| | | Example | | Example | | Example | |
|---|---|---|---|---|---|---|---|
| | | 1.1 | | 1.2 | | 1.3 | |
| | | | % by | | % by | | % by |
| | Constituent | (mg) | wt. | (mg) | wt. | (mg) | wt. |
| A | Sevelamer HCl | 800 | 77.0 | 800 | 83.5 | 800 | 91.1 |
| B | Lactose | 200 | 19.3 | 100 | 10.4 | | |
| C | Highly disperse | 10 | 1.0 | 14 | 1.5 | 17 | 1.9 |
| | silicon dioxide | | | | | | |
| D | Croscarmellose | 21 | 2.0 | 34 | 3.6 | 43 | 4.9 |
| E | Magnesium stearate | 7 | 0.7 | 10 | 1.0 | 18 | 2.1 |
| | Total | 1038 | 100.0 | 958 | 100.0 | 878 | 100.0 |

The procedure in Example 1.1. was carried out as described below.

A mixture of Sevelamer hydrochloride with a density of 1.26 g/cm³ and lactose was prepared using a Lödige MTG30 high-speed mixer by intensively mixing 6.4 kg of Sevelamer hydrochloride, 1.6 kg of lactose in the mixer for 10 min. This mixture was then slugged on a roll compacter suitable for pharmaceutical purposes with a gap width of 2 mm and crushed over a breaking sieve with mesh width 1.0 mm. The resulting crushed compact (=granules) was mixed with highly disperse silicon dioxide and croscarmellose after sieving, and end-mixed with magnesium stearate. Following compression on a high-performance rotary tableting press to give tablets with a pregiven size (formulation 1.1: oblong 20.0 by 9.4 mm, height 7.2 mm), the in-process controls customary for the medicament form were carried out.

The mass proved to be readily tabletable, no sticking or capping occurred, the results of the in-process controls were within the customary range, e.g. but not exclusively hardness 121-145 newtons, disintegration time 9-10 min.

Examples 1.2 and 1.3 were carried out analogously.

It was likewise possible to achieve tablets with a hardness greater than 120 N and a disintegration time of less than 10 minutes when Sevelamer hydrochloride with a water content of ca. 4% by weight or alternatively 9% by weight was used.

### Examples 2.1 to 2.3

### Tablets which are Swallowed in Unchewed Form

The following tablets were produced in accordance with the method of the invention.

| | | Example | | Example | | Example | |
|---|---|---|---|---|---|---|---|
| | | 2.1 | | 2.2 | | 2.3 | |
| | | | % by | | % by | | % by |
| | Constituent | (mg) | wt. | (mg) | wt. | (mg) | wt. |
| A | Sevelamer carbonate | 800 | 92.6 | 800 | 87.1 | 800 | 81.5 |
| B | Copolyvidone | 30 | 3.5 | 60 | 6.5 | 94 | 9.6 |
| C | Highly disperse | 4 | 0.45 | 4 | 0.4 | 9 | 0.9 |
| | silicon dioxide (1) | | | | | | |
| D | Stearic acid (1) | 5 | 0.6 | 5 | 0.5 | 10 | 1.0 |
| E | Highly disperse | 4 | 0.45 | 10 | 1.1 | 9 | 0.9 |
| | silicon dioxide (2) | | | | | | |
| F | Crospovidone | 16 | 1.8 | 32 | 3.5 | 45 | 4.6 |
| G | Stearic acid (2) | 5 | 0.6 | 8 | 0.9 | 15 | 1.5 |
| | Total | 864 | 100.0 | 919 | 100.0 | 982 | 100.0 |

Examples 2.1 to 2.3 were carried out as described below.

A mixture of Sevelamer carbonate (essentially free from Sevelamer hydrochloride) and copolyvidone was prepared using a high-speed mixer. Then, in a free-fall mixer, highly disperse silicon dioxide (1) and stearic acid (1) were mixed in after sieving and the mixture was compacted on a roll compacter suitable for pharmaceutical purposes. After passing over a crushing sieve with 1.5 mm, the crushed compact obtained was mixed with highly disperse silicon dioxide (2) and crospovidone after sieving and end-mixed with stearic acid (2). Following compression on a high-performance rotary tableting press to give tablets of pregiven size (formulation 2.1. Oblong 22.0 by 9.4 mm, height 5.8 mm), the in-process controls customary for the medicament form were carried out.

The mass proved to be readily tabletable, no sticking or capping occurred, the results of the in-process controls were within the desired range. Thus, for a formulation as in Example 2.1, for example, a hardness of 100-123 newtons and a disintegration time of 4 to 7 min was established.

Moreover, the tablets as in Example 2 were stored for 3 weeks. Following storage, the tablets have a disintegration time of less than 15 minutes.

The tablets according to Examples 1 and 2 could optionally be coated with a customary aqueous or aqueous-alcoholic film.

For this, hypromellose was prepared with water, following dissolution admixed with talc, polyethylene glycol and titanium dioxide and this suspension was coated on in a perforated drum coater:

Moisture in the cores before coating: 2.7%
Moisture in the film tablets after coating: 2.8%
Mass of the film coating: 28 mg per tablet
Hardness of the film tablets from Example 1: 135-170 N
Disintegration time of the film tablets from Example 1: 17-23 min.

None of the film-coated tablets exhibited flakes.

### Examples 3.1 to 3.4

### Tablets which are Swallowed in Unchewed Form

The following tablets were prepared in accordance with the method of the invention.

| | | | | Example | Example |
|---|---|---|---|---|---|
| | | Example 3.1 | Example 3.2 | 3.3 | 3.4 |
| | Constituent | (mg) | (mg) | (mg) | (mg) |
| A | Sevelamer | 800 | 800 | 800 | 800 |
| | hydrochloride | | | | |
| B | Highly disperse | 9 | 9 | 9 | 9 |
| | silicon dioxide | | | | |
| C | Kollidon ® 30 | 63 | 63 | — | — |
| D | Starlac ® | — | — | 63 | — |
| E | Isomalt | — | — | — | 63 |
| F | Crospovidone | 18 | 18 | 18 | 18 |
| G | Sodium stearyl | 9 | 9 | 9 | 9 |
| | fumarate | | | | |
| | Total | 899 | 899 | 899 | 899 |

The tablets were produced essentially as described in Example 1 and 2. In Example 3.1, however, only Sevelamer was compacted together with highly disperse silicon dioxide whereas in Examples 3.2 to 3.4 Sevelamer was compacted together with highly disperse silicon dioxide and binder (Kollidon®, Starlac® or isomalt).

For the average breaking strength, the friability and the disintegration, the following values arise:

| | Example | Example | Example | Example |
|---|---|---|---|---|
| Value | 3.1 | 3.2 | 3.3 | 3.4 |
| Average breaking | 163 | 164 | 154 | 167 |
| strength (N) | | | | |
| Friability after | 1.2 | 0.4 | 1.4 | 0.9 |
| 20 min (%) | | | | |
| Disintegration in | 3.45 | 3.22 | 0.55 | 1.31 |
| water (min, sec) | | | | |

### Examples 4.1 to 4.3

### Dispersible Tables

The following dispersible tablets were produced in accordance with the method of the invention.

| | | Example | | Example | | Example | |
|---|---|---|---|---|---|---|---|
| | | 4.1 | | 4.2 | | 4.3 | |
| | | | % by | | % by | | % |
| | Constituent | (mg) | wt. | (mg) | wt. | (mg) | by wt. |
| A | Sevelamer HCl | 800 | 61.3 | 800 | 57.8 | 800 | 55.0 |
| B | Microcrystalline | 150 | 11.5 | 200 | 14.4 | 250 | 17.2 |
| | cellulose (1) | | | | | | |
| C | Highly disperse | 13 | 1.0 | 17.5 | 1.3 | 17.5 | 1.2 |
| | silicon dioxide (1) | | | | | | |
| D | Sodium stearyl | 13 | 1.0 | 4 | 0.3 | 28 | 1.9 |
| | fumarate (1) | | | | | | |
| E | Highly disperse | 13 | 1.0 | 17.5 | 1.3 | 17.5 | 1.2 |
| | silicon dioxide (2) | | | | | | |
| F | Microcrystalline | 200 | 15.3 | 200 | 14.4 | 200 | 13.8 |
| | cellulose (2) | | | | | | |
| G | Croscarmellose | 65 | 5.0 | 100 | 7.2 | 85 | 5.8 |
| H | Aspartame | 13 | 1.0 | 13 | 0.9 | 13 | 0.9 |
| I | Saccharin sodium | 13 | 1.0 | 13 | 0.9 | 13 | 0.9 |
| J | Aroma | 13 | 1.0 | 13 | 0.9 | 13 | 0.9 |
| K | Sodium stearyl | 13 | 1.0 | 7 | 0.5 | 17.5 | 1.2 |
| | fumarate (2) | | | | | | |
| | Total | 1306 | 100.0 | 1385 | 100.0 | 1454.5 | 100.0 |

A mixture of Sevelamer hydrochloride and microcrystalline cellulose (1) was prepared using a high-speed mixer. Then, in a free-form mixer, highly disperse silicon dioxide (1) and sodium stearyl fumarate (1) were added after sieving and the mixture was compacted on a roll compacter suitable for pharmaceutical purposes. After passing over a crushing sieve 1.25 mm, the resulting crushed compact was mixed with highly disperse silicon dioxide (2) and croscarmellose after sieving, then end-mixed with sodium stearyl fumarate (2) and then compressed on a high-performance rotary tableting press to give tablets of pregiven size (for formulation 4.1 round, biplane, diameter 18 mm, height 5.5 mm).

The mass proved to be readily tabletable, no sticking or capping occurred, the results of the in-process controls were within the desired range. Thus, for the formulation as in Example 4.3, for example a hardness of 90-145 newtons and a disintegration time of 3-5 min in 200 ml of water at room temperature were established.

### Examples 5.1 and 4.2

### Chewable Tablets

The following chewable tablets were produced in accordance with the method of the invention.

| | | Example 5.1 | | Example 5.2 | |
|---|---|---|---|---|---|
| | | | % by | | % by |
| | Constituent | (mg) | wt. | (mg) | wt. |
| A | Sevelamer HCl | 800 | 42.1 | 800 | 50.7 |
| B | Mannitol (1) | 300 | 15.8 | 200 | 12.7 |
| C | Highly disperse silicon dioxide (1) | 19 | 1.0 | 19 | 1.2 |
| D | Adipic acid (1) | 19 | 1.0 | 7 | 0.4 |
| E | Highly disperse silicon dioxide (2) | 19 | 1.0 | 19 | 1.2 |
| F | Pregelatinized starch | 150 | 7.9 | 150 | 9.5 |
| G | Aspartame | 19 | 1.0 | 19 | 1.2 |
| H | Saccharin sodium | 19 | 1.0 | 19 | 1.2 |
| I | Aroma | 36 | 1.9 | 36 | 2.4 |
| J | Mannitol (2) | 500 | 26.3 | 300 | 19.0 |
| K | Adipic acid (2) | 19 | 1.0 | 8 | 0.5 |
| | Total | 1900 | 100.0 | 1577 | 100.0 |

A mixture of Sevelamer hydrochloride and mannitol (1) was prepared using a high-speed mixer. Then, in a free-fall mixer, highly disperse silicon dioxide (1) and adipic acid (1) were mixed in after sieving and the mixture was compacted on a roll compacter suitable for pharmaceutical purposes. After passing over a crushing sieve with a mesh width of 1.0 mm, the resulting crushed compact was mixed with highly disperse silicon dioxide (2), pregelatinized starch, mannitol (2), saccharin Na, aspartame and aroma after sieving, end-mixed with adipic acid (2) and then compressed on a high-performance rotary tableting press to give tablets of pregiven size (for formulation 5.1 round, biplane, diameter 20 mm, height 5.4 mm).

The mass proved to be readily tabletable; the results of the in-process controls were within the desired range. Thus, for formulation 5.1, for example the hardness at 70-105 N was within the readily chewable range; the abrasion was 0.37%.

### Examples 6.1 and 6.2

### Granules for Pouring into Sachets

The following granules were produced in accordance with the method of the invention.

| | | Example 6.1 | | Example 6.2 | |
|---|---|---|---|---|---|
| | | | % by | | % by |
| | Constituent | (mg) | wt. | (mg) | wt. |
| A | Sevelamer HCl | 800 | 73.8 | 800 | 52.2 |
| B | Maltitol (1) | 175 | 16.1 | 250 | 16.3 |
| C | Pregelatinized starch | 50 | 4.6 | 150 | 9.8 |
| D | Highly disperse silicon dioxide (1) | 11 | 1.0 | 11 | 0.7 |
| E | Polyethylene glycol 6000 | 5 | 0.5 | 20 | 1.3 |
| F | Highly disperse silicon dioxide (2) | 11 | 1.0 | 11 | 0.7 |
| G | Maltitol (2) | | | 250 | 16.3 |
| H | Aspartame | 11 | 1.0 | 14 | 0.9 |
| I | Saccharin sodium | 11 | 1.0 | 14 | 0.9 |
| J | Aroma | 11 | 1.0 | 14 | 0.9 |
| | Total | 1085 | 100.0 | 1534 | 100.0 |

A mixture was prepared from Sevelamer HCl, maltitol (1) and pregelatinized starch using a high-speed mixer. Then, in the free-fall mixer, highly disperse silicon dioxide (1) and polyethylene glycol 6000 (pulverulent) were mixed in after sieving and the mixture was compacted on a roll compacter suitable for pharmaceutical purposes. After passing over a crushing sieve with a mesh width of 0.8 mm, the resulting crushed compact was mixed with maltitol (2), highly disperse silicon dioxide (2), saccharin Na, aspartame and aroma after sieving and poured into sachets.

The mass proved to be readily pourable; the relative standard deviation of the fill masses for formulation 6.1 was srel=4.2 to 4.6%.

1. A method of producing tablets comprising a polyallylamine polymer, comprising the steps
(i) providing a polyallylamine polymer or pharmaceutically compatible salts thereof, optionally in a mixture with one or more pharmaceutical excipients;
(ii) compaction to give a slug;
(iii) granulation of the slug; and
(iv) compression of the resulting granules to give tablets.

2. The method as claimed in claim 1, where the compaction conditions in step (ii) are selected such that the slug has a density of 1.18 to 1.50 g/cm³, preferably 1.20 to 1.30 g/cm³.

3. The method as claimed in claim 1 characterized in that the compaction is carried out in a roll granulator.

4. The method as claimed in claim 3, where the gap width of the roll granulator is 1 to 3 mm.

5. The method as claimed in claim 3 where the rolling force is 2 to 20 kN/cm, preferably 3 to 15 kN/cm.

6. The method as claimed in claim 1 where the granulation conditions in step (iii) are selected such that the resulting particles have a weight-average particle size of from 50 μm to 500 μm.

7. The method as claimed in claim 1 where the granulation takes place in a sieving mill with a mesh width of the sieve insert of from 0.75 mm to 2 mm.

8. The method as claimed in claim 1 characterized in that, in step (i),
(a) 65 to 99% by weight of polyallylamine polymer or pharmaceutically compatible salts thereof and
(b) 1 to 30% by weight of pharmaceutically compatible excipients are mixed.

9. The method as claimed in claim 1 where process step (iv) is chosen such that the tablets produced comprise at least 800 mg of a polyallylamine polymer or pharmaceutically compatible salts thereof.

10. The method as claimed in claim 1 where the tablet is additionally covered with a film in a step (v).

11. The method as claimed in claim 1 characterized in that the polyallylamine polymer is Sevelamer, Colesevelam or pharmaceutically compatible salts thereof.

12. The method as claimed in claim 11, characterized in that the polyallylamine polymer is Sevelamer hydrochloride and/or Sevelamer carbonate.

13. The method as claimed in claim 12, characterized in that a multiple compaction takes place, where the granules resulting from step (iii) are returned one or more times to the compaction (ii).

14. A tablet comprising a polyallylamine polymer or pharmaceutically compatible salts thereof, obtainable by a method as claimed in claim 1.

15. A tablet comprising a polyallylamine polymer, where the tablet has a bimodal pore size distribution.

16. The tablet as claimed in claim 14 which is a tablet which is swallowed in unchewed form, a chewable tablet or a dispersible tablet.

17. A slug comprising a polyallylamine polymer, obtainable by a method comprising the steps
(i) providing a polyallylamine polymer or pharmaceutically compatible salts thereof, optionally in a mixture with one or more pharmaceutical excipients;
(ii) compaction to give a slug.

18. Granules, in particular for filling sachets, comprising a polyallylamine polymer, obtainable by a method comprising the steps
(i) providing a polyallylamine polymer or pharmaceutically compatible salts thereof, optionally in a mixture with one or more pharmaceutical excipients;
(ii) compaction to give a slug; and
(iii) granulation of the slug.

19. The method as claimed in claim 1 where a polyallylamine polymer with a density greater than 1.24 g/cm³, preferably with a density of from 1.25 to 1.30 g/cm³, is used.
